(19) United States
(12) Patent Application Publication
(10) Pub. No.: US 2024/0216683 A1
(43) Pub. Date: Jul. 4, 2024

(54) MEDICAL DEVICE AND METHOD FOR DETERMINING AN ORIENTATION OF SAME

(71) Applicant: BIOTRONIK SE & Co. KG, Berlin (DE)

(72) Inventors: Shayan Guhaniyogi, Portland, OR (US); R. Hollis Whittington, Portland, OR (US); Dirk Muessig, West Linn, OR (US); Ravi Kiran Kondama Reddy, Portland, OR (US); Pamela Shamsie Victoria Riahi, Portland, OR (US)

(21) Appl. No.: 18/563,039

(22) PCT Filed: May 2, 2022
(86) PCT No.: PCT/EP2022/061702

§ 371 Date: Nov. 21, 2023

Related U.S. Application Data
(60) Provisional application No. 63/193,668, filed on May 27, 2021.

(30) Foreign Application Priority Data
Jul. 14, 2021 (EP) 21185518.4

(51) Int. Cl. A61N 1/36; A61N 1/02; A61N 1/365; A61N 1/39

(52) U.S. Cl. CPC A61N 1/36031; A61N 1/025; A61N 1/36135; A61N 1/36542; A61N 1/3925

(57) ABSTRACT

A medical device for implantation within or mounting on a patient's body includes an accelerator unit, a data memory unit and a processor which are electrically interconnected. Three orthogonal axes (XD, YD, ZD) are defined for the medical device and three orthogonal axes (XP, YP, ZP) are defined for the patient's body. The accelerator unit is configured to determine 3-dimensional proper acceleration data along sensitive axes corresponding to the three orthogonal axes of the medical device (XD, YD, ZD). In order to provide an automatic estimation of medical device orientation with regard to a patient's body (thereby overcoming the drawbacks of manual calibration methods) which does not make a priori assumptions about the device's orientation or specific prevalence, the processor is configured to process said acceleration data determined by the accelerator unit.

The invention is directed to a medical device for implantation within or mounted on a patient's body. Such medical device is usually configured to monitor the health condition of a patient and/or to deliver a therapy to the patient. The invention is further directed to a method for determining an orientation of same, a respective computer program product and a respective computer readable data carrier.

An active or passive medical device, for example, a pacemaker (with leads), a BioMonitor, an Implantable Leadless Pacer (ILP), an Implantable Leadless Pressure Sensor (ILPS), an Implantable Cardiac Defibrillator (ICD) or a Shockbox, a device that delivers spinal cord stimulation (SCS), deep brain stimulation (DBS) or neurostimulation or a device that delivers one or more therapeutic substances, e.g. a drug pump, contains sensors that collect physiological signals in order to monitor the health status of the patient and/or delivers a therapy to the patient.

As patient monitoring in general hospital wards and at home becomes more and more common, the circumstances, under which the monitored parameters are obtained or under which the therapy is delivered, have to be assessed automatically and with increasing accuracy.

With regard to health condition monitoring and therapy application, patient posture is an important clinical measure which can be affected by, and therefore may be an indicator for, an improvement or decline in the patient's health condition. Implantable or body-mountable devices containing accelerometers are capable of determining the posture of the patient implanted with the device, but only if the orientation of the device with respect to the patient's body is known. Therefore, determining the orientation of the medical device with respect to the patient's body is a critical step in providing a patient posture metric.

Known medical devices either require a manual calibration to determine the device orientation with regard to the patient's body, or they perform automatic estimation of the orientation using assumptions which generally may not hold true.

Manual calibration methods for determining the device orientation with regard to the patient's body require a procedure in which the patient assumes various positions while the medical device communicates with a device programmer. First, this procedure may be cumbersome and not desired by either the patient or health care provider (HCP). Second, as a medical device may migrate in location over time, manual calibration methods would necessitate re-performing this procedure at regular intervals (e.g. during patient follow-ups) in order to keep correct estimates of the device orientation and thereby the patient's posture.

Existing automatic estimations of device orientation with regard to a patient's body use algorithms that make the assumption that the “yaw” angle of the device relative to the patient is close to zero, wherein yaw is defined as the angle between the device's Z axis and the Z axis of the patient's body. While this may sometimes be a safe assumption for devices implanted in the pectoral region, it is not always guaranteed. Furthermore, devices implanted in other regions of the body may have a “yaw” angle which differs significantly from zero depending on the specific region. Therefore, the existing solutions make an assumption which is not generalizable and may not even be true for the originally intended scope of the solution (i.e. pectoral device implants). As a result, the estimated device orientations and patient postures obtained by existing automatic solutions may be incorrect.

Document EP 2 598 028 B1 describes a method for automatic calibration of an orientation of an implanted or body-mounted device relative to the body. In order to assess the accelerometer data the method defines a reference condition that comprises a reference posture or reference posture range which has at least one predetermined characteristic. The characteristic relates to a particularly high or low prevalence of this posture or posture range which may be incorrect in certain circumstances. Further, the automatic calibration uses cluster analysis which is computationally intensive. Additionally, the known method assumes that the patient's supine posture is perfectly flat.

Document U.S. Pat. No. 8,758,274 describes a method that creates posture state definitions, each including a defined vector describing a corresponding posture state based on the coordinate system of an accelerometer or a gyroscope of a medical device. The orientation of the patient's body with regard to this coordinate system is not considered.

Accordingly, it is desirable to provide an automatic estimation of medical device orientation with regard to a patient's body (thereby overcoming the drawbacks of manual calibration methods) which does not make a priori assumptions about the device's orientation or specific prevalence and therefore is generalizable to medical devices implanted or body-mounted in any region of the body.

The above problem is solved by a medical device with the features of claim 1, by a method for determining an orientation of a medical device with the features of claim 7, by a computer program product with the features of claim 13, and by a computer readable data carrier with the features of claim 14.

In particular, the problem is solved by a medical device for implantation within or mounting on a patient's body comprising an accelerator unit, a data memory unit and a processor which are electrically interconnected, wherein for the medical device three orthogonal axes (XD, YD, ZD) and for the patient's body three orthogonal axes (XP, YP, ZP) are defined, wherein the accelerator unit is configured to determine 3-dimensional proper acceleration data along sensitive axes corresponding to the three orthogonal axes of the medical device (XD, YD, ZD) and the processor is configured to process said acceleration data determined by the accelerator unit, wherein the processor is configured
to differentiate between an active state and a rest state of the patient's body,
to determine an actual orientation of the three orthogonal axes of the medical device (XD, YD, ZD) with regard to a horizontal plane and/or to determine an actual yaw-angle between one axis ZD of the medical device and the corresponding axis ZP of the patient's body,
wherein, in order to determine the actual orientation of the three orthogonal axes of the medical device (XD, YD, ZD) with regard to a horizontal plane the processor is configured
   to receive a first group of said acceleration data determined by the accelerator unit within a first predefined time interval that lies immediately after the time point for which the processor identifies the transition from the active state to the rest state of the patient's body and/or to receive a second group of said acceleration data determined by the accelerator unit within a second predefined time interval that lies immediately before the time point for which the processor identifies the transition from the rest state to the active state of the patient's body and to calculate at least one specific acceleration data from the first group of acceleration data and/or the second group of acceleration data and transfer the at least one specific acceleration data to the data memory unit for storage,
   to read from the data memory unit the specific acceleration data from all groups of said acceleration data that were determined by the accelerator unit within a third predefined time interval, and
   to determine the actual orientation of the three orthogonal axes of the medical device (XD, YD, ZD) with regard to the horizontal plane based on the specific acceleration data,
wherein, in order to determine the actual yaw-angle between the one axis ZD of the medical device and the corresponding axis ZP of the patient's body the processor is configured
   to determine whether the patient's body performs a predefined specific activity in its active state,
   to receive said acceleration data continuously determined by the accelerator unit over a predefined fourth time interval during performance of the predefined specific activity by the patient's body, and
   to determine the actual yaw-angle between the one axis ZD of the medical device and the corresponding axis ZP of the patient's body based on the continuously determined acceleration data of the fourth time interval.

The medical device is any device which is configured to be implanted partly or fully within a patient's body or mounted on the patient's body. As indicated above the medical device is configured to monitor the health condition of a patient and/or to deliver a therapy to the patient. Further, as an implanted or body-mounted device the medical device moves with the patient's body as it is thereby basically fixed to the body. In some cases, the medical device may carry out a very small relative movement with regard to the patient's body if the patient moves due to the tissue properties but this will be ignored in the following. Particularly, the medical device is a pacemaker, ILP, ICD, Shockbox, device that delivers SCD, or a BioMonitor. The invention may also be used in the above examples of medical devices. Mounting on the patient's body means that the medical device is permanently or temporarily attached to the patient's body, for example by suturing.

For the medical device three orthogonal axes (XD, YD, ZD) and for the patient's body three orthogonal axes (XP, YP, ZP) are defined. Each system of three orthogonal axes (XD, YD, ZD) and (XP, YP, ZP) forms a 3-dimensional Cartesian coordinate system, wherein the axes XD and XP are an x-axis, the axes YD and YP a y-axis and the axes ZD and ZP a z-axis. With regard to a pacemaker, the axis ZD is the axis which is normal to the largest flat surfaces of the pacemaker. Accordingly, the largest flat surfaces are parallel to the XD, YD-plane. Regarding the patient's body the +XP axis is towards the patient's left arm, the +YP axis is towards the patient's head, and the +ZP axis is outwards from the patient's chest. When the patient is in a standing position, this means that the patient +XP, +YP, and +ZP axes are orthogonal, anti-parallel, and the axes XP, ZP orthogonal to the earth's direction of gravitation, respectively.

The medical device comprises the accelerator unit, the data memory unit and the processor which are electrically interconnected. This means, inter alia, that they are capable to exchange data.

The accelerator unit of the medical device may comprise a 3-dimensional accelerometer sensor, for example one 3-dimensional accelerometer or several 1- or 2-dimensional accelerometers, such as a piezo electric and/or micro-electro mechanical (MEMs) accelerometer and/or mechanical accelerometer and/or gravimeter or any combination of these sensors. The term 3-dimensional means that the acceleration unit is configured to determine 3-dimensional proper acceleration data along sensitive axes corresponding to the three orthogonal axes of the medical device (XD, YD, ZD). Accordingly, the acceleration data determined by the acceleration unit always comprise three values, namely the acceleration along the XD-axis, the acceleration along the YD-axis and the acceleration along the ZD-axis. Proper acceleration is the acceleration (the rate of change of velocity) of the unit in its own instantaneous rest frame. For example, an accelerometer at rest on the surface of the Earth will measure an acceleration into the direction of Earth's gravity, straight upwards (by definition) of g≈9.81 m/s² because the Earth's surface exerts a normal force upwards relative to the local inertial frame (the frame of a freely falling object near the surface). By contrast, accelerometers in free fall (falling toward the center of the Earth at a rate of about 9.81 m/s²) will measure zero. Usually, the acceleration is quantified in the SI unit m/s² or in terms of standard gravity (multiples of g).

The data memory unit of the medical device may include any volatile, non-volatile, magnetic, or electrical media, such as a random-access memory (RAM), read-only memory (ROM), non-volatile RAM (NVRAM), electrically-erasable programmable ROM (EEPROM), flash memory, or any other memory device. The data memory unit may store the measured (raw) acceleration data determined by the acceleration unit.

With regard to the invention the processor is regarded as a functional unit of the medical device, that interprets and executes instructions comprising an instruction control unit and an arithmetic and logic unit. The processor may comprise a microprocessor, a controller, a digital signal processor (DSP), an application specific integrated circuit (ASIC), a field-programmable gate array (FPGA, discrete logic circuitry or any combination thereof. The processor processes the (raw) acceleration data received from the accelerator unit directly or the acceleration data received from the data memory unit.

The medical device may comprise further modules such a power supply such as a battery, at least one signal generator for generating, for example, electrical or electromagnetically therapy signals in order to provide the therapy to the patient, a sender module for sending data to an external unit such as a remote computer or Programmer and/or a transceiver module for bi-directionally exchanging data with such remote computer or Programmer. The sender transceiver module, the power supply, the at least one sensor and/or the signal generator may be electrically connected to the processor. All above mentioned modules/units may be modules/units separate from the processor or may be at least partly integrated within the processor of the medical device. The sender module and/or the transceiver module may communicate wirelessly with the remote computer or Programmer, for example using electromagnetic waves, for example Bluetooth, WLAN, ZigBee, NFC, Wibree or WiMAX in the radio frequency region, or IrDA or free-space optical communication (FSO) in the infrared or optical frequency region. Communication by wire (electrical and/or optical communication) may be possible, as well. The remote computer is a functional unit that can perform substantial computations, including numerous arithmetic operations and logic operations without human intervention, such as, for example, a personal mobile device (PMD), a desktop computer, a server computer, clusters/warehouse scale computer or embedded system.

The processor is configured to differentiate between an active state and a rest state of the medical device and thereby of the patient's body as the medical device moves with the patient's body. According to the invention, the patient's body is in an active state if the processor determines an actual activity/motion level (of the medical device) above a predefined threshold. If the activity level is below or equal to this threshold, it is assumed that the patient's body is at rest. The activity level may be determined using different sensors, for example the accelerator unit, other sensors such as a gyroscope, heart rate sensor, temperature sensor, or respiration sensor. If the activity level is determined using the accelerator unit, the processor processes the 3-dimensional acceleration data of the acceleration unit in real time with the intent of determining patient activity (i.e. motion). As an example, the device may process (e.g. filter, rectify, integrate, etc.) the output of one axis (or multiple axes) and then compare the processed value to a predefined threshold. If the processed value is greater than the predefined threshold, the device will indicate that the patient's body is in the active state. The specific processing steps and threshold may be defined such that the device will only detect patient motion/activity during events that are generally accepted to be physical activities, such as walking, running, cycling, (stair) climbing, jumping up and down, and will not trigger during events that are not generally accepted to be physical activities, such as breathing.

According to the invention the processor is adapted to determine an actual orientation of the three orthogonal axes of the medical device (XD, YD, ZD) with regard to a horizontal plane (first component of orientation determination) and/or to determine an actual yaw-angle between one axis ZD of the medical device and the corresponding axis ZP of the patient's body (second component of orientation determination). After determination of both components of orientation determination, i.e. of the actual orientation of the three orthogonal axes of the medical device (XD, YD, ZD) with regard to a horizontal plane and of the actual yaw-angle between the axes ZD, ZP, the orientation of all axes of the medical device (XD, YD, ZD) with regard to all axes of the patient's body (XP, YP, ZP) is known. However, one component may be known quite perfectly so that it is only necessary to determine the other component thereby receiving the full actual orientation of the medical device and the patient's body.

According to the invention the processor is configured to determine the actual orientation (θₓ, θ_{y}, θ_{z}) of the three orthogonal axes of the medical device (XD, YD, ZD) when the patient is upright with regard to the horizontal plane as shown in FIG. 7, i.e. the plane orthogonal to the gravity direction (which is independent of the medical device and the patient's body), wherein θₓ is the angle enclosed by the actual position of axis XD and the horizontal plane, θ_{y} is the angle enclosed by the actual position of axis YD and the horizontal plane and θ_{z} is the angle enclosed by the actual position of axis ZD and the horizontal plane. In order to determine the orientation (θₓ, θ_{y}, θ_{z}) the processor monitors the activity of the patient's body as indicated above. If the processor identifies a transition from the active state to the rest state, the processor receives a first group of 3-dimensional acceleration data determined by the accelerator unit within a first predefined time interval that lies immediately after the time point for which the processor identifies the transition from the active state to the rest state of the patient's body. “Immediately” means that the first time interval may begin directly or within a short time interval (e.g. less than a second) after this time point. The first time interval, in which the first group of acceleration data is measured may have the length of several seconds. Additionally or alternatively, the processor receives a second group of the 3-dimensional acceleration data determined by the accelerator unit within a second predefined time interval that lies immediately before the time point for which the processor identifies the transition from the rest state to the active state of the patient's body. “Immediately” means that the second time interval may end directly or within a short time interval (e.g. less than a second) prior to this time point. The second time interval, in which the second group of acceleration data is measured may have the length of several seconds. The above procedure is based on the key insight that most daily living events generally accepted to be physical activities are performed in an upright, and often standing, position. Therefore, the majority of patient activities that the medical device detects should be activities which are performed when the patient is in an upright standing position. If this is the case, it is therefore appropriate to assume that immediately before and after the activity, the patient is upright, standing, and at rest. Consequently, the 3-dimensional acceleration values measured by the accelerator unit immediately before or after such activity corresponds to the acceleration data when the patient's body is upright, standing, and at rest. From the first group of acceleration data and/or from the second group of acceleration data at least one specific acceleration data is calculated, for example an average of the respective group of acceleration data, wherein the average may be the arithmetic mean, the geometric mean or the harmonic mean value. The average is determined from the acceleration values of the respective group for each axis separately. In this embodiment the specific acceleration data is one 3-dimensional average acceleration value. This specific acceleration data (in the described embodiment one average 3-dimensional acceleration value for the respective group) is then transferred to and stored in the data memory unit of the device. Each specific acceleration data is regarded as a snapshot of the medical device's orientation for the first time interval or for the second time interval, respectively.

Then, at the end of the third predefined time interval (e.g. several hours, days or weeks) the specific acceleration data from all groups of said acceleration data that were determined by the accelerator unit within the third predefined time interval (which is longer than or equal to the first time interval and longer than or equal to the second time interval) are read by the processor from the data memory unit. All specific acceleration data form at least one or a plurality of snapshots. Practically speaking, not all of these snapshots will truly capture when the patient's body is upright and at rest. As examples, the device may trigger activity during an event in which the patient is not upright, or the patient may change posture from/to a non-upright position immediately before/after an upright activity. However, these instances would typically be in the minority of snapshots captured. Accordingly, in one embodiment from these read specific acceleration data of third time interval a median acceleration data or another average acceleration data is then determined. The advantage of determining the median acceleration data is that the above outliers do not influence the median result. The median acceleration value or the average acceleration value is determined from the specific acceleration data as a median value or an average value (e.g. the mentioned mean values above) for each axis separately, wherein each of the median acceleration data or the average acceleration data is a 3-dimensional acceleration data. In case the third time interval covers only one specific acceleration data, the average acceleration data or the median acceleration data corresponds to the respective specific acceleration data.

Finally, from the specific acceleration data, the median acceleration data or the average acceleration data the actual orientation of the three orthogonal axes of the medical device (XD, YD, ZD) with regard to a horizontal plane, when the patient is upright, may be determined using the following equations Eq. 1 to 3:

$${θ}_{x} = {tan}^{- 1} \frac{{a}_{x}}{\sqrt{{a}_{y}^{2} + {a}_{z}^{2}}} \qquad ( Eq . 1 )$$

$${θ}_{y} = {tan}^{- 1} \frac{{a}_{y}}{\sqrt{{a}_{x}^{2} + {a}_{z}^{2}}} \qquad ( Eq . 2 )$$

$${θ}_{z} = {tan}^{- 1} \frac{{a}_{z}}{\sqrt{{a}_{x}^{2} + {a}_{y}^{2}}} , \qquad ( Eq . 3 )$$

wherein aₓ, a_{y}, a_{z} are the acceleration data components for each axis of the specific acceleration data, the median acceleration data or the average acceleration data.

Further, the actual yaw-angle between the one axis ZD of the medical device and the corresponding axis ZP of the patient's body is determined by the processor. The yaw-angle is denoted here as Φ and is defined as the angle between the medical device's Z axis (i.e. ZD) and the Z axis of the patient's body (i.e. the anteroposterior axis of the patient, ZP), as shown in FIG. 9. Note that if the patient's body is upright and stationary, any change in Φ (while (θₓ, θ_{y}, θ_{z}) are kept fixed) does not change the accelerator unit output. It is therefore not possible to calculate Φ using just the accelerator unit output when the patient is standing.

For determining the actual yaw-angle the following steps are executed by the processor of the medical device:
determining whether the patient's body performs a predefined specific activity in its active state, for example a walking activity,
receiving said acceleration data continuously determined by the accelerator unit over a predefined fourth time interval during performance of the predefined specific activity by the patient's body,
determining the actual yaw-angle between the one axis ZD of the medical device and the corresponding axis ZP of the patient's body based on the continuously determined acceleration data of the third time interval, for example using signal-processing techniques to extract time- and frequency-domain features for any combination of accelerometer axes.

The above procedure is executed because it was observed that the acceleration data measured by the acceleration unit during the specific activity changes if the yaw angle Φ changes. Accordingly, the yaw-angle can be derived from the acceleration data continuously measured over the predefined fourth time interval (for example several seconds to several minutes) for the specific activity. The measurement is stopped if the specific activity is stopped. If another activity type is used for determining the yaw-angle, the determination procedure needs to be adapted to the other activity type.

Determination of the predefined specific activity can be achieved by using advanced signal processing techniques on the accelerometer data, such as, for example, a machine learning classifier that has been trained to identify various predefined activities from accelerometer data. Alternatively, because the most common activity of daily living is walking, whenever an active state is detected by the device, the device may calculate the yaw-angle, assuming that the activity is a walking activity, and store it to memory, resulting in a plurality of stored yaw-angles. Note that for the calculation of the yaw-angle, the assumption that all activities are a walking activity will be generally incorrect, and therefore there will be some incorrect yaw-angle calculations. However, for most populations the majority of activities are walking activities, and therefore the majority of calculated yaw-angles will be correct. Therefore, after some sixth time period (e.g. a day, a week, etc.) the device may calculate the median of the stored yaw-angles, and this median result will produce the correct yaw-angle due to the nature of the median calculation and the majority of stored yaw-angles being correct.

Calculation of the yaw-angle for a walking activity is achieved as follows. During a walking activity, acceleration along the XP axis has a fundamental frequency that is half the fundamental frequency along the YP or ZP axes, due to the nature of the walking motion (gait). The fundamental frequency in the YP and ZP axes are equal to the step frequency (i.e. number of steps/second) of the walking activity. For example, if the patient walks at 2 steps/second (2 Hz), the fundamental frequency of acceleration along the YP and ZP axes will be equal to the step frequency of 2 Hz, while the fundamental frequency of acceleration along the XP axis will be equal to half the step frequency, 1 Hz.

Note that a yaw-angle Φ of 0° indicates that the ZD axis of the device is aligned with the ZP axis of the patient, while a Φ of 90° indicates that the ZD axis of the device is aligned with the XP axis of the patient. Consequently, the fundamental frequency of acceleration data from the ZD axis of the device will be equal to the step frequency when Φ=0°, but will be equal to half the step frequency when Φ=90°. Therefore, it is possible to determine Φ by examining the frequency-domain features of the ZD axis and comparing them to the step frequency of the walking activity.

The step frequency of the walking activity can be calculated from the accelerometer data collected by the device in several ways. As one particular example, the device may use the previously calculated (θₓ, θ_{y}, θ_{z}) to determine which axis is most aligned with the YP axis of the patient, corresponding to a θ=90°. For example, if the (θₓ, θ_{y}, θ_{z}) were found to be (0°, 80°, 10°), then the device would determine that the YD axis is most aligned with the YP axis of the patient because θ_{y} (80°) is closest to 90°. Finally, the device would calculate the fundamental frequency of the accelerometer data collected from the YD axis during the walking activity to determine the step frequency. As another example, the device may calculate the total acceleration from all three axes during the walking activity, using the following equation:

$${a}_{total} = \sqrt{{a}_{x}^{2} + {a}_{y}^{2} + {a}_{z}^{2}} , \qquad ( Eq . 4 )$$

Finally, the device would calculate the fundamental frequency of aₜₒₜₐₗ to determine the step frequency.

Once the step frequency is known, the frequency-domain features of accelerometer data from the ZD axis can be examined to determine the yaw-angle. As one example, the signal power at the step frequency (Pₛₜₑₚ) and the signal power at half the step frequency (P_{halfstep}) can be calculated. As described earlier, when the ZD axis is aligned with ZP (Φ=0°) the fundamental frequency of the acceleration from the ZD axis will be equal to the step frequency, meaning that the bulk of signal power from the acceleration data will be around the step frequency, and Pₛₜₑₚ will be much larger than P_{halfstep}. Conversely, when the ZD axis is aligned with XP (Φ=90), the fundamental frequency of the acceleration from the ZD will be equal to half the step frequency, meaning that the bulk of signal power from the acceleration data will be around half the step frequency, and Pₛₜₑₚ will be much smaller than P_{halfstep}.

Therefore, an estimate of Φ can be calculated by assessing the relationship between the measured Pₛₜₑₚ and P_{halfstep}. The relationship may be, for example, a learned regression model relating Pₛₜₑₚ and P_{halfstep} to Φ from a dataset of accelerometer signals during walking from many patients. As another example, the relationship may be as simple as the ratio of Pₛₜₑₚ to P_{halfstep}. The former approach is likely to be more accurate, however the specific relationship used by a particular device to calculate Φ will be dependent on the accuracy required by such device and the computational resources available to the device.

The above is only one particular example of how to estimate Φ during a walking activity. However, other time- and/or frequency-domain methods can also be used, including but not limited to relationships between signal amplitudes or magnitudes, signal-to-noise ratios, phase, etc. These relationships can be calculated among any of the accelerometer axes in the device. Such relationships must be calculated based on knowledge of the activity during which Φ is calculated: the above described a walking activity, but other activities may also be used if sufficient knowledge about the expected relationships in the accelerometer data is known for those particular activities.

In one embodiment the data memory module comprises a circular buffer configured such that acceleration data defined above are continuously stored in the circular buffer for a predefined time interval, for example several seconds up to several minutes wherein acceleration data which are older than this time interval are overwritten by the newest acceleration data. Using the circular buffer memory space is saved.

In one embodiment, as explained below in more detail, the processor is configured to determine an actual posture of the patient based on the determined actual orientation of the three orthogonal axes of the medical device (XD, YD, ZD) with regard to the horizontal plane and the determined actual yaw-angle between one axis ZD of the medical device and the corresponding axis ZP of the patient's body. The determined patient posture may be used to calculate the percentage of time the patient is upright versus in laying down as an overall health status indicator. Further, the determined posture may be utilized to corroborate other indicators of the patient's health, for example (e.g., respiratory effort, cough indicator, fever indicator). The patient's posture is the position in which the full body of the patient is. There are several postures as the patient goes about daily life, for example upright or horizontal postures like lying forward or prone (face down), lying backward or supine (face up), lying right, lying left, etc.

The above problem is also solved by a method for determining an orientation of a medical device for implantation within or mounting on a patient's body comprising an accelerator unit, a data memory unit and a processor which are electrically interconnected, wherein for the medical device three orthogonal axes (XD, YD, ZD) and for the patient's body three orthogonal axes (XP, YP, ZP) are defined, wherein the accelerator unit determines 3-dimensional proper acceleration data along sensitive axes corresponding to the three orthogonal axes of the medical device (XD, YD, ZD) and the processor processes said acceleration data determined by the accelerator unit, wherein the processor
differentiates between an active state and a rest state of the patient's body,
determines an actual orientation (θₓ, θ_{y}, θ_{z}) of the three orthogonal axes of the medical device (XD, YD, ZD) with regard to a horizontal plane and/or determines an actual yaw-angle Φ between one axis of the medical device ZD and the corresponding axis of the patient's body ZP,
wherein the actual orientation of the three orthogonal axes of the medical device (XD, YD, ZD) with regard to a horizontal plane is determined by the processor with the following steps
   receiving a first group of said acceleration data determined by the accelerator unit within a first predefined time interval that lies immediately after the time point for which the processor identifies the transition from the active state to the rest state of the patient's body and/or receiving a second group of said acceleration data determined by the accelerator unit within a second predefined time interval that lies immediately before the time point for which the processor identifies the transition from the rest state to the active state of the patient's body and calculating at least one specific acceleration data from the first group of acceleration data and/or the second group of acceleration data and transferring the at least one specific acceleration data to the data memory unit for storage,
   reading from the data memory unit the at least one specific acceleration data from all groups of said acceleration data that were determined by the accelerator unit within a third predefined time interval, and
   determining the actual orientation of the three orthogonal axes of the medical device (XD, YD, ZD) with regard to the horizontal plane based on the at least one specific acceleration data,
wherein the actual yaw-angle Φ between the one axis ZD of the medical device and the corresponding axis ZP of the patient's body is determined by the processor with the following steps
   determining whether the patient's body performs a predefined specific activity in its active state, for example walking,
   receiving said acceleration data continuously determined by the accelerator unit over a predefined fourth time interval during performance of the predefined specific activity by the patient's body,
   determining the actual yaw-angle Φ between the one axis ZD of the medical device and the corresponding axis ZP of the patient's body based on the continuously determined acceleration data of the third time interval.

In one embodiment of the above method the at least one specific acceleration data derived from the first group of acceleration data and/or the second group of acceleration data is an average of the respective group of acceleration data.

In one embodiment of the above method from all read at least one specific acceleration data of the third time interval a median acceleration data is determined and the actual orientation of the three orthogonal axes of the medical device (XD, YD, ZD) with regard to the horizontal plane is calculated from the determined median acceleration data.

In one embodiment said acceleration data are continuously stored in a circular buffer for a predefined fifth time interval, wherein acceleration data which are older than the fifth time interval are overwritten by the newest acceleration data.

Once (θₓ, θ_{y}, θ_{z}) and Φ are known, the orientation of the medical device with respect to the patient's body is fully-known, and the processor of the medical device may thereafter accurately determine the patient's posture at any time based on these values. More specifically, in order to the determine the patient's posture the medical device may thereafter measure the accelerator unit output at any time that the patient is inactive. The medical device may then calculate the orientation of the device with respect to the horizontal plane at this particular instant in time, using Eqs. 1-3. These new angles can be called (αₓ, αₓ and α_{z}). Any difference between (αₓ, αₓ and α_{z}) and (θₓ, θ_{y}, θ_{z}) corresponds to a difference in the patient's posture compared to a standing position (because (θₓ, θ_{y}, θ_{z}) was calculated from a standing reference posture, as described above). This difference between (αₓ, αₓ and α_{z}) and (θₓ, θ_{y}, θ_{z}), and knowledge of Φ (which does not change) therefore fully describes the patient's new posture relative to a standing position. Note that calculating the difference between (αₓ, αₓ and α_{z}) and (θₓ, θ_{y}, θ_{z}), and the use of Φ in the manner described above can be achieved by using standard methods well-known in the field of linear algebra, such as rotation matrices, dot-products, etc., and are therefore not elaborated upon further here.

Once the patient's posture is estimated, it can be used in various indicators of the patient's health. One such indicator may be the percentage of time the patient is upright versus in a “laying down” position. For example, if a daily trend of the patient's posture indicates that the percentage of time spent laying down is increasing, it may be used with other device sensors/metrics (e.g. temperature, activity) to screen for fever.

The posture estimation may also be used for more specific trends, such as night-time posture only. As an example, a night-time posture trend may be tracked when a patient is going from a supine lying position to a more upright lying position over the course of a few days in order to relieve pulmonary congestion. In general, changes in night-time posture may reveal changes the patient is making in order to sleep/breathe more comfortably. The posture trend may also be paired with a respiratory effort algorithm that measures respiratory rate/tidal volume algorithm (using an impedance or accelerometer signal) to further corroborate changes in breathing ability.

As another example, if the device has a cough detection algorithm (e.g., using an impedance or accelerometer signal), the posture estimation may be used to corroborate this algorithm: an increase in coughs and a change in the patient's posture trend may provide more evidence of a respiratory infection to an HCP. The changes in the posture trend may occur either throughout the day, or at specific times. For example, a transition to more upright postures during the night only may be used to corroborate a respiratory infection.

The above method is, for example, realized as a computer program which comprises instructions which, when executed, cause the processor to perform the steps of the above method (to be executed by the medical device, in particular at its processor) which is a combination of above and below specified computer instructions and data definitions that enable computer hardware to perform computational or control functions or which is a syntactic unit that conforms to the rules of a particular programming language and that is composed of declarations and statements or instructions needed for a above and below specified function, task, or problem solution.

Furthermore, a computer program product is disclosed comprising instructions which, when executed by a processor, cause the processor to perform the steps of the above defined method. Accordingly, a computer readable data carrier storing such computer program product is disclosed.

The technical advantage of this invention is that it provides the ability to estimate Φ automatically without a manual calibration procedure, and without assuming it is equal to zero, thereby leading to higher accuracy of the determined posture of the patient. The determination of the actual values (θₓ, θ_{y}, θ_{z}) and Φ of a medical device may be used for many clinical applications, including heart failure monitoring, improved activity recognition/classification algorithms, and objective measures of pain and health outcomes. It also has significant clinical utility in the current COVID-19 pandemic, providing a potentially important indicator of the patient's overall health and a means to corroborate other indicators of the disease. Furthermore, the technique is applicable to many products (see above examples) and may improve existing or upcoming activity recognition/classification algorithms.

The present invention will now be described in further detail with reference to the accompanying schematic drawing, wherein

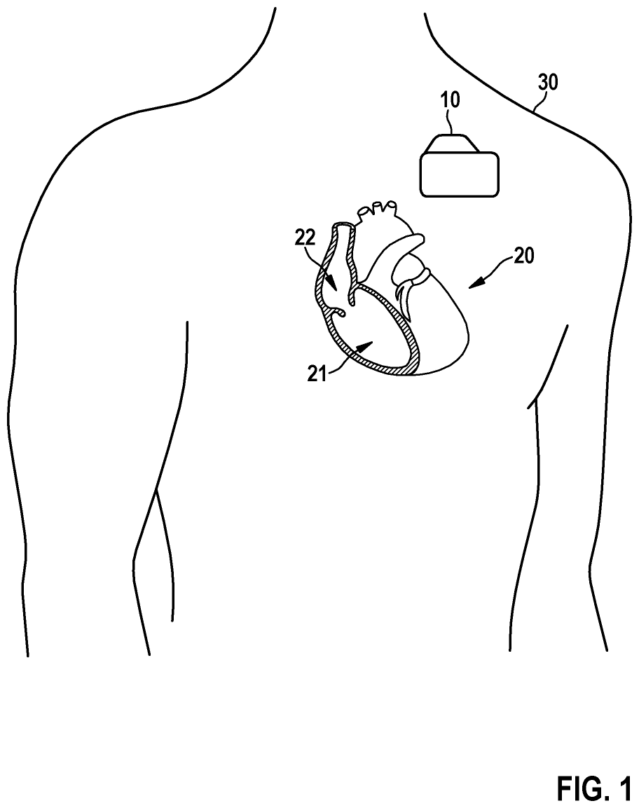

FIG. 1 shows an embodiment of the inventive medical device implanted within a patient's body,

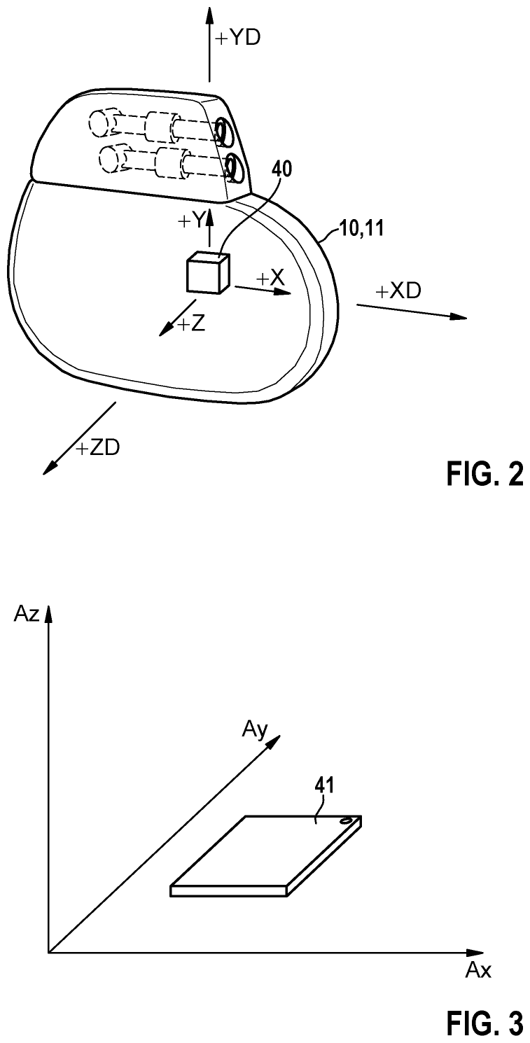

FIG. 2 depicts the inventive medical device of FIG. 1 a perspective side view,

FIGS. 3 to 5 show the sensitive axes of an accelerator unit with regard to the gravity direction of the medical device of FIG. 1 in perspective side views,

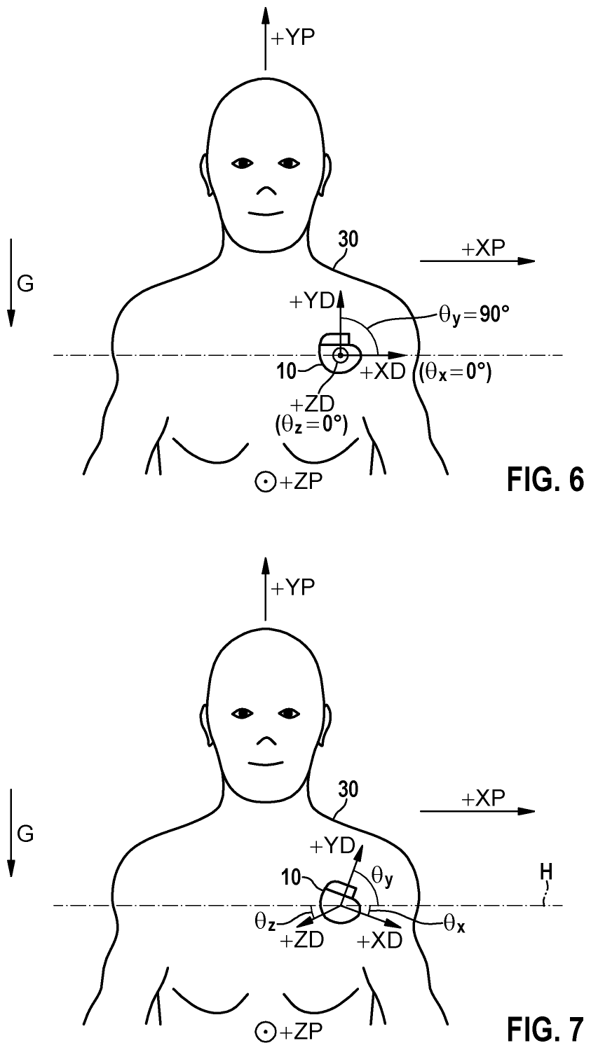

FIG. 6 shows the medical device of FIG. 1, the ideal orientation of the axes of this medical device with regard to the horizontal plane and the patient's body in a front view,

FIG. 7 depicts the medical device of FIG. 1, another orientation of the axes of this medical device with regard to the horizontal plane and the patient's body in a front view,

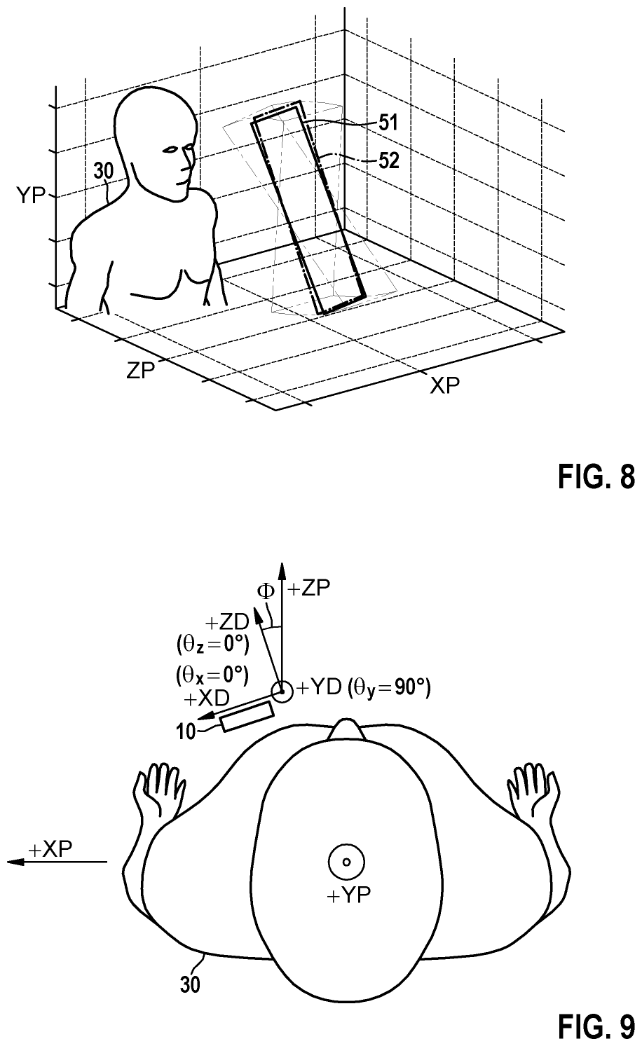

FIG. 8 shows an example of results for the actual orientation of the three orthogonal axes of the medical device with regard to the horizontal plane in comparison with the actual orientation measured otherwise with the patient's body in a perspective side view,

FIG. 9 depicts the determination of the yaw angle with the patient's body and the medical device in a top view,

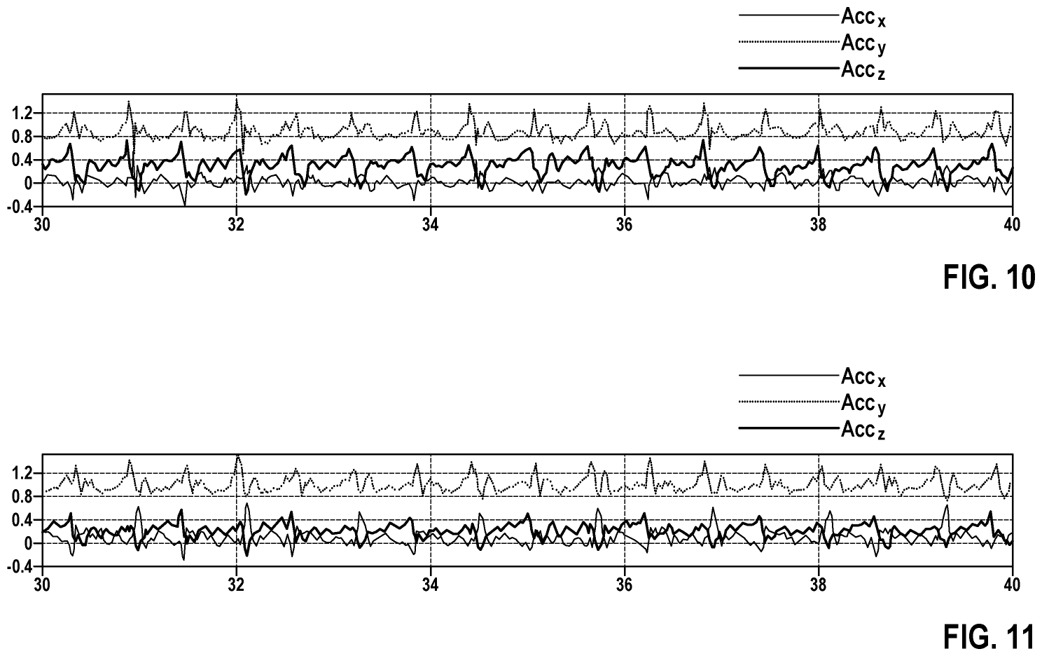

FIG. 10 an example of acceleration data over time for a walking activity and a yaw-angle Φ=0°,

FIG. 11 an example of acceleration data over time for a walking activity and a yaw-angle Φ=45°, and

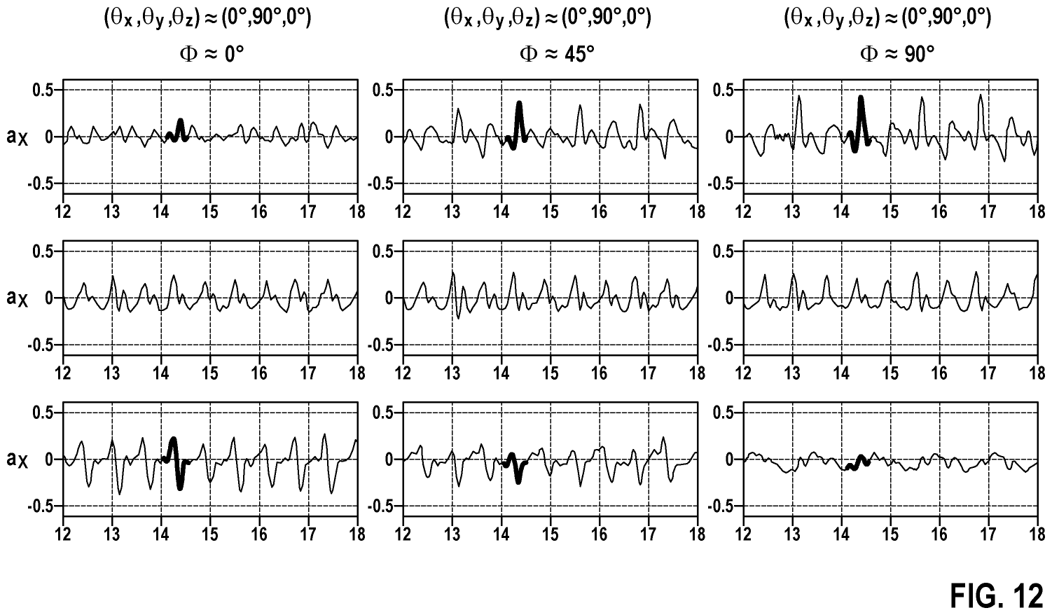

FIG. 12 another example of acceleration data over time for a walking activity and a yaw-angle Φ=0° (first column), Φ=45° (second column) and Φ=90° (third column).

FIG. 1 shows an example medical device in form of a pacemaker 10 and a heart 20 (with right ventricle 21 and right atrium 22). The invention may be realized in other medical devices analogously. The pacemaker 10 is implanted within a patient's body 30 at its pectoral region. The pacemaker 10 may be configured to pace the right ventricle 21 and the right atrium 22 of the heart 20 in a well-known manner. The pacemaker 10 further comprises an accelerator unit 40 shown in FIGS. 2 and 3 to 5. The axes of a 3-dimensional orthogonal coordinate system XD, YD and ZD of the pacemaker 10 are shown in FIG. 2. From this FIG. one may derive that the sensitive axes X, Y and Z of the accelerator unit 40 are aligned with the axes XD, YD and ZD, respectively of the pacemaker 10. The accelerator unit 40 is mounted in the pacemaker 10 such that its Z-axis (and the pacemaker axis ZD) is normal to the flat surface of a housing 11 pacemaker 10. The accelerator unit 40 is located within the housing 11 of the pacemaker 10 and is hermetically sealed within the housing 11.

The pacemaker 10 may comprise modules/units (not shown) such as a processor, a data memory unit, a signal generator unit for providing treatment signals (e.g. pacing signals), a sensor comprising an IEGM measuring unit for sensing ventricular depolarization, a transceiver module for sending and receiving messages to a Programmer or remote computer, and a power source wherein the modules/units are electrically connected to each other. The power source may include a battery, e.g., a rechargeable or non-rechargeable battery. The data memory unit may include any memory type mentioned above.

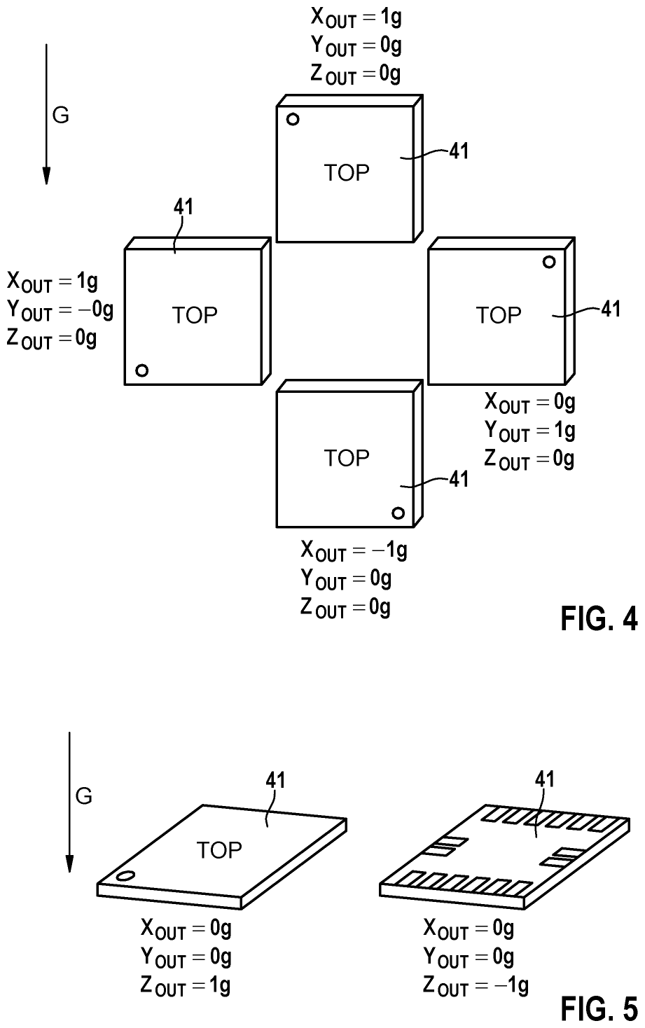

The accelerator unit 40 comprises an accelerometer sensor 41, for example ultralow power MEMS accelerometer ADXL362 of Analog Devices, which is sensitive to both DC and AC accelerations in the three orthogonal directions depicted in FIG. 2 with X, Y and Z. The DC component of accelerations experienced by the sensor is due to the gravitational field. Assuming there is no AC component of acceleration (i.e. there is no dynamic movement) the sensor will produce specific outputs when aligned with the gravitational field in various orientations, as demonstrated in FIGS. 3 to 5. FIG. 3 shows the axes of acceleration sensitivity Ax, Ay, Az along which the corresponding output increases when accelerated along the respective sensitive axis. FIGS. 4 and 5 depict the output (Xout, Yout, Zout) of the sensor with regard to each axis when positioned in different orientations to gravity (see arrow G).

For a pacemaker 10 implanted in the chest of the patient, the ideal scenario from an orientation perspective is when the pacemaker's XD, YD, and ZD axes are aligned with the patient's body XP, YP, and ZP axes. The patient's body +XP axis is towards the patient's left arm, the +YP axis is towards the patient's head, and the +ZP axis is outwards from the patient's chest. When the patient is in a standing position, this means that the patient's body +XP, +YP, and +ZP axes are orthogonal, and the XP, ZP-plane is further orthogonal to the earth's gravitational field direction G, respectively.

FIG. 6 illustrates the ideal orientation of the pacemaker's axes (XD, YD, ZD) and patient's body axes (XP, YP, ZP) with respect to the earth's gravitational field, when the patient is standing. If the patient were standing perfectly upright, the angles between the device axes (XD, YD, ZD) and a plane normal to the to the earth's gravitational field direction G, i.e. the horizontal plane H depicted by a dashed line in FIG. 6, would be (θₓ, θ_{y}, θ_{z})=(0°, 90°, 0°), and the sensor outputs of the device in this scenario would be (ax, ay, az)=(0g, 1g, 0g). The sensor outputs are also referred to as acceleration data.

Typically, due to implantation considerations the pacemaker's and patient's body axes will not align in the ideal orientation depicted in FIG. 6. A more typical scenario is shown in FIG. 7. In this figure, the pacemaker 10 has been rotated from its ideal position: specifically, it has been rotated about the patient's body XP and ZP axes, thereby changing the angles (θₓ, θ_{y}, θ_{z}) between the device axes and the horizontal plane H.

It is now explained, how the processor of the pacemaker 10 determines the actual orientation of the three orthogonal axes of the pacemaker (XD, YD, ZD) with regard to the horizontal plane H. First, the processor processes the outputs (ax, ay, az) of the accelerator unit 40 derived by its sensor 41 in real time with the intent of determining patient activity (i.e. motion). As an example, the device could process (e.g. filter, rectify, integrate, etc.) the output of one axis (or multiple axes) and then compare the processed value to a pre-defined threshold. If the processed value is greater than the threshold, the device will indicate the presence of patient activity. Similarly, once the pacemaker 10 has flagged that the patient is in a state of motion, the device can wait for the processed value to fall below the threshold in order to indicate that the patient has stopped performing an activity and is at rest. The specific processing steps and threshold can be defined such that the device will only detect patient motion during events that are generally accepted to be physical activities, such as walking, and will not trigger during events that are not generally accepted to be physical activities, such as breathing.

For example, the sensor 41 output (ax, ay, az) can be stored continuously in a circular buffer of the data memory unit for several seconds, overwriting old data as new data comes in. When the processor triggers that patient activity has occurred (using the method described above), the data in the buffer covering a second time interval (e.g. several seconds) immediately prior to the trigger can be averaged and stored in RAM of the data memory unit. Alternatively, or in addition to this, when the processor triggers that patient activity has ceased, the buffer may be allowed to fill for another time interval (first time interval) of several seconds. The acceleration data for each of the first time interval and the second time interval are then averaged and stored in RAM of the data memory unit. Both methods effectively store many snapshots in the form of one triple of acceleration data for each first time interval or second time interval of the sensor output when the patient is assumed to be standing and at rest.

Then, the median of all the “standing” snapshots stored in RAM may be used to approximate the sensor output for the patient in an upright standing position. Specifically, at the end of the day the device may calculate the median value of all the “standing” snapshots stored in RAM throughout one day (third time interval). Then this median value of acceleration data for each pacemaker axis is used to calculate the angles (θₓ, θ_{y}, θ_{z}) according to above equations Eq. 1, Eq. 2 and Eq. 3. This means that the median values are used as aₓ, a_{y}, a_{z-values} and from the Eq. 1 to 3 the values (θₓ, θ_{y}, θ_{z}) are determined. The third time interval can be extended (e.g. to several days or weeks) to further improve the reliability of the calculation.

The results of a prototype implementation of the above algorithm is shown in FIG. 8. A device containing an accelerometer was placed on a human subject and data was collected for 24 hours. To obtain the “true” angles, the patient was asked to stand upright for a certain period of time during which data was recorded, thus allowing calculation of the true (θx, θy, θz). For the remainder of the 24 hours, the patient performed activities of daily living without any supervision and without prescribed changes to their typical daily activities. The box 51 in FIG. 5 shows the “true” device orientation using the human-annotated data in which the patient was asked to stand upright. The dashed boxes show all the device orientations calculated from the “standing” snapshots described above during the 24-hour data collection period. The box 52 shows the final estimated orientation of the pacemaker provided by the algorithm after calculating the median of all of the snapshots, which shows strong agreement to the true orientation (box 51). Note that for the boxes drawn in FIG. 8, the “yaw” angle of the pacemaker (described below) is assumed to be zero.

In addition to calculating the angles (θx, θy, θz) of the pacemaker's axes with respect to the horizontal plane H, their angles with respect to the patient's body 30 must also be calculated for a full description of the device orientation. Once (θx, θy, θz) is known, only one more angle is needed for this full description, which will be called “yaw” to maintain consistency with terminology in prior art. Yaw, denoted here as Φ, can be defined as the angle between the pacemaker's axis ZD and the patient's body axis ZP (i.e. the anteroposterior axis of the patient's body), as shown in FIG. 9.

For the purpose of an example, consider a walking activity as a specific activity of the active state. FIGS. 10 and 11 shows accelerometer data of the sensor 41 collected from a pacemaker 10 implanted within a patient's body 30 during a walking activity over time. In this example, the pacemaker's orientation was approximately (θx, θy, θz)˜=(0°, 90°, 0°) and Φ˜=0° in FIG. 10. FIG. 11 shows accelerometer data of the sensor 41 over time collected simultaneously during the same walking activity from a different pacemaker 10 which was oriented approximately (θx, θy, θz)˜=(0°, 90°, 0°) and Φ˜=45° (i.e. Φ was the primary difference between the two pacemakers' orientations).

Based on the differences between FIGS. 10 and 11, it is apparent that the features of an accelerometer signal ax, ay, az during walking for each accelerometer/pacemaker axis may change as Φ changes. A more detailed look at these changes is shown in FIG. 12, which demonstrates how the features of a accelerometer signal during walking over time changes for each axis as Φ is varied from ˜0° to ˜45° to ˜90°. In each of these examples (θx, θy, θz) was approximately (0°, 90°, 0°). Note that the DC signal offset is removed from each axis (using a high-pass filter) to better compare the changes in the time-varying signal for each axis.

FIG. 12 demonstrates that dynamic signal features, including amplitude and frequency content, may change in a particular set of axes as the yaw angles Φ changes (see FIG. 12 captions of each column for detailed description). Based on these findings, an estimate of Φ may be calculated as follows. After the algorithm for calculating (θx, θy, θz) is complete, the pacemaker 10 stores accelerometer data when a particular activity of interest is detected. As an example, the processor of pacemaker 10 may employ an algorithm to detect a walking activity, and store accelerometer data during a fourth time period of several seconds of this activity. The device will then use appropriate signal processing techniques to calculate time- and/or frequency-domain features for each axis, or combination of axes. Any of these features and the relationships among them may then be used to estimate Φ. As one example, FIG. 12 demonstrates that during walking the amplitudes in certain axes change as Φ changes: this provides a simple signal-processing technique to estimate Φ that is not computationally-intensive.

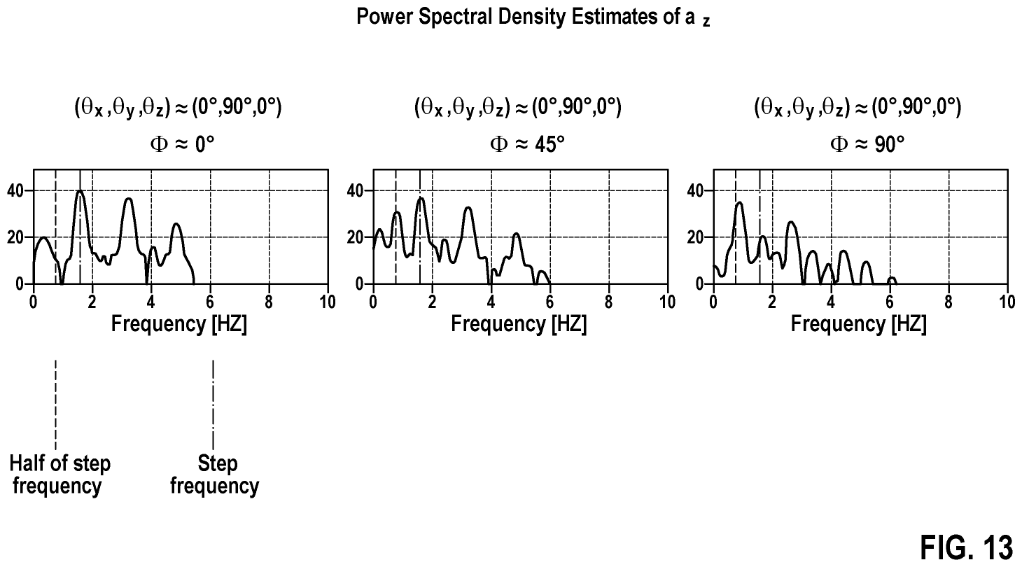

As another example, FIG. 12 also demonstrates that as Φ changes from 0° to 90°, the step frequency component in the az signal decreases, while the frequency component equal to half of the step frequency becomes more visible. FIG. 13 shows the power spectral density estimates of the az signals shown in FIG. 12. It can be seen that the power at the step frequency decreases as Φ changes from 0 to 90°, while the power at half the step frequency increases. Therefore the relationship between the powers at the step frequency and half the step frequency can also be used to estimate Φ.

Another example on how to calculate Φ during a walking activity is achieved as follows. During a walking activity, acceleration along the patient's X axis has a fundamental frequency that is half the fundamental frequency along the patient's Y or Z axes, due to the nature of the walking motion (gait). The fundamental frequency in the Y and Z axes are equal to the step frequency (i.e. number of steps/second) of the walking activity. For example, if the patient walks at 2 steps/second (2 Hz), the fundamental frequency of acceleration along the patient's Y and Z axes will be equal to the step frequency of 2 Hz, while the fundamental frequency of acceleration along the patient's X axis will be equal to half the step frequency, 1 Hz.

Note that a Φ of 0° indicates that the Z axis of the device is aligned with the Z axis of the patient, while a Φ of 90° indicates that the Z axis of the device is aligned with the X axis of the patient. Consequently, the fundamental frequency of acceleration data from the Z axis of the device will be equal to the step frequency when Φ=0°, but will be equal to half the step frequency when Φ=90. Therefore, it is possible to determine Φ by examining the frequency-domain features of the accelerometer data from the device's Z axis and comparing them to the step frequency of the walking activity.

Similar to the calculation of (θx, θy, θz) described above, the processor of the pacemaker 10 may calculate and store Φ many times over a given time period (e.g. hours, days, weeks) and then perform an aggregation function (e.g. median) to reduce the influence of outliers and provide a reliable estimate of Φ.

Once (θx, θy, θz) and Φ are known, the orientation of the pacemaker 10 with respect to the patient's body 30 is fully-known, and the pacemaker 10 can thereafter accurately estimate the patient's posture at any time. More specifically, the pacemaker 10 can thereafter measure the accelerometer sensor output while the patient is inactive, apply an appropriate transformation to the output using the estimated (θx, θy, θz) and Φ, and use the transformed output to estimate the patient's posture.

1. A medical device (10) for implantation within or mounting on a patient's body (30) comprising an accelerator unit (40), a data memory unit and a processor which are electrically interconnected, wherein for the medical device three orthogonal axes (XD, YD, ZD) and for the patient's body three orthogonal axes (XP, YP, ZP) are defined, wherein the accelerator unit is configured to determine 3-dimensional proper acceleration data along sensitive axes corresponding to the three orthogonal axes of the medical device (XD, YD, ZD) and the processor is configured to process said acceleration data determined by the accelerator unit, wherein the processor is configured to:
(i) differentiate between an active state and a rest state of the patient's body; and
(ii) determine an actual orientation (θₓ, θ_{y}, θ_{z}) of the three orthogonal axes of the medical device (XD, YD, ZD) with regard to a horizontal plane (H) or determine an actual yaw-angle (F) between one axis (ZD) of the medical device and the corresponding axis (ZP) of the patient's body;
wherein, in order to determine the actual orientation (θₓ, θ_{y}, θ_{z}) of the three orthogonal axes of the medical device (XD, YD, ZD) with regard to a horizontal plane (H) the processor is configured to:
(i) receive a first group of said acceleration data determined by the accelerator unit within a first predefined time interval that lies immediately after the time point for which the processor identifies the transition from the active state to the rest state of the patient's body or receive a second group of said acceleration data determined by the accelerator unit within a second predefined time interval that lies immediately before the time point for which the processor identifies the transition from the rest state to the active state of the patient's body and to calculate at least one specific acceleration data from the first group of acceleration data or the second group of acceleration data and transfer the at least one specific acceleration data to the data memory unit for storage,
(ii) read from the data memory unit the specific acceleration data from all groups of said acceleration data that were determined by the accelerator unit within a third predefined time interval, and
(iii) determine the actual orientation of the three orthogonal axes of the medical device (XD, YD, ZD) with regard to the horizontal plane based on the specific acceleration data; and
wherein, in order to determine the actual yaw-angle (F) between the one axis (ZD) of the medical device and the corresponding axis (ZP) of the patient's body the processor is configured to:
(i) determine whether the patient's body performs a predefined specific activity in its active state,
(ii) receive said acceleration data continuously determined by the accelerator unit over a predefined fourth time interval during performance of the predefined specific activity by the patient's body, and
(iii) determine the actual yaw-angle between the one axis ZD of the medical device and the corresponding axis ZP of the patient's body based on the continuously determined acceleration data of the fourth time interval.

2. The medical device of claim 1, wherein the at least one specific acceleration data derived from one or both of the first group of acceleration data or the second group of acceleration data is an average of the respective group of acceleration data.

3. The medical device of claim 1, wherein from all read at least one specific acceleration data of the third time interval a median acceleration data is determined and the actual orientation (θₓ, θ_{y}, θ_{z}) of the three orthogonal axes of the medical device (XD, YD, ZD) with regard to the horizontal plane (H) is calculated from the determined median acceleration data.

4. The medical device of claim 1, wherein the data memory unit is configured such that said acceleration data are continuously stored in a circular buffer for a predefined fifth time interval, wherein acceleration data which are older than the fifth time interval are overwritten by the newest acceleration data.

5. The medical device of claim 1, wherein the specific activity is walking.

6. The medical device of claim 1, wherein the processor is configured to determine an actual posture of the patient based on the determined actual orientation (θₓ, θ_{y}, θ_{z}) of the three orthogonal axes of the medical device (XD, YD, ZD) with regard to the horizontal plane (H) and the determined actual yaw-angle (F) between one axis (ZD) of the medical device and the corresponding axis (ZP) of the patient's body (30).

7. A method for determining an orientation of a medical device (10) for implantation within or mounting on a patient's body (30) comprising an accelerator unit (40), a data memory unit and a processor which are electrically interconnected, wherein for the medical device three orthogonal axes (XD, YD, ZD) and for the patient's body three orthogonal axes (XP, YP, ZP) are defined, wherein the accelerator unit determines 3-dimensional proper acceleration data along sensitive axes corresponding to the three orthogonal axes of the medical device (XD, YD, ZD) and the processor processes said acceleration data determined by the accelerator unit, wherein the processor:
differentiates between an active state and a rest state of the patient's body;
(ii) determines one or both of the following:
(A) an actual orientation (θₓ, θ_{y}, θ_{z}) of the three orthogonal axes of the medical device (XD, YD, ZD) with regard to a horizontal plane (H), or
(B) an actual yaw-angle (F) between one axis of the medical device (ZD) and the corresponding axis (ZP) of the patient's body;
wherein the actual orientation (θₓ, θ_{y}, θ_{z}) of the three orthogonal axes of the medical device (XD, YD, ZD) with regard to a horizontal plane is determined by the processor with the following steps:
(i) receiving one or both of the following:
(A) a first group of said acceleration data determined by the accelerator unit within a first predefined time interval that lies immediately after the time point for which the processor identifies the transition from the active state to the rest state of the patient's body
(B) a second group of said acceleration data determined by the accelerator unit within a second predefined time interval that lies immediately before the time point for which the processor identifies the transition from the rest state to the active state of the patient's body and calculating at least one specific acceleration data from one or both of the first group of acceleration data or the second group of acceleration data and transferring the at least one specific acceleration data to the data memory unit for storage,
(ii) reading from the data memory unit the at least one specific acceleration data from all groups of said acceleration data that were determined by the accelerator unit within a third predefined time interval, and
(iii) determining the actual orientation of the three orthogonal axes of the medical device (XD, YD, ZD) with regard to the horizontal plane based on the at least one specific acceleration data; and
wherein the actual yaw-angle (F) between the one axis (ZD) of the medical device and the corresponding axis (ZP) of the patient's body is determined by the processor with the following steps:
(i) determining whether the patient's body performs a predefined specific activity in its active state,
(ii) receiving said acceleration data continuously determined by the accelerator unit over a predefined fourth time interval during performance of the predefined specific activity by the patient's body, and
(iii) determining the actual yaw-angle between the one axis ZD of the medical device and the corresponding axis ZP of the patient's body based on the continuously determined acceleration data of the third time interval.

8. The method of claim 7, wherein the at least one specific acceleration data derived from the one or both of the first group of acceleration data or the second group of acceleration data is an average of the respective group of acceleration data.

9. The method of claim 7, wherein from all read at least one specific acceleration data of the third time interval a median acceleration data is determined and the actual orientation (θₓ, θ_{y}, θ_{z}) of the three orthogonal axes of the medical device (XD, YD, ZD) with regard to the horizontal plane (H) is calculated from the determined median acceleration data.

10. The method of claim 7, wherein said acceleration data are continuously stored in a circular buffer for a predefined fifth time interval, wherein acceleration data which are older than the fifth time interval are overwritten by the newest acceleration data.

11. The method of claim 7, wherein the specific activity is walking.

12. The method of claim 7, wherein an actual posture of the patient is determined by the processor based on the determined actual orientation (θₓ, θ_{y}, θ_{z}) of the three orthogonal axes of the medical device (XD, YD, ZD) with regard to the horizontal plane (H) and the determined actual yaw-angle (F) between one axis (ZD) of the medical device and the corresponding axis (ZP) of the patient's body (30).

13. A computer program product comprising instructions which, when executed by a processor, cause the processor to perform the steps of the method according to claim 7.

14. A computer readable data carrier storing a computer program product according to claim 13.
